(19) United States
(12) Plant Patent Application Publication
(10) Pub. No.: US 2013/0081184 P1
(43) Pub. Date: Mar. 28, 2013

(54) Heucherella plant named 'Solar Eclipse'

(50) ;

(71) Applicants: Janet N. Egger, Wilsonville, OR (US); John C. Pavlich, East Olympia, WA (US)

(72) Inventors: Janet N. Egger, Wilsonville, OR (US); John C. Pavlich, East Olympia, WA (US)

(73) Assignee: Terra Nova Nurseries, Inc.

(21) Appl. No.: 13/200,474

(22) Filed: Sep. 22, 2011

(51) Int. Cl. A01H 5/00

(52) U.S. Cl. PLT/441

(57) ABSTRACT

A new and distinct Heucherella plant substantially as shown and described, characterized by large red to brown leaves with a yellow to lime margin, leaves which are palmately lobed, scalloped, and slightly ruffled, a high crown count, a tight, mounding plant habit, and excellent vigor. It grows equally well in full sun or filtered shade in the Pacific Northwest.

### BOTANICAL DENOMINATION

Heucherella

### VARIETY DESIGNATION

‘Solar Eclipse’

Cross reference to Heucherella ‘Solar Power’ U.S. Plant patent application Ser. No. 12/806,901

### BACKGROUND OF THE INVENTION

The present invention relates to a new and distinct cultivar of Heucherella and given the cultivar name of ‘Solar Eclipse’. Heucherella is in the family Saxifragaceae. Heucherella ‘Solar Eclipse’ originated from a tissue culture mutation from Heucherella ‘Solar Eclipse’.

### SUMMARY OF THE INVENTION

Compared to the seed parent Heucherella ‘Solar Power’, the new variety has a higher crown count, a tighter mounding habit, more rounded leaf lobes, a more ruffled leaf margin, and a much darker leaf center pattern which encompasses the whole leaf except the edge.

This new Heucherella is uniquely distinguished by:

1. large red to brown leaves with a yellow to lime margin,

2. palmately lobed leaves which are scalloped, slightly ruffled,

3. a high crown count,

4. a tight, mounding plant habit,

5. grows equally well in full sun or filtered shade in the Pacific Northwest, and

6. excellent vigor.

This new cultivar has been reproduced only by asexual propagation (division and tissue culture). Each of the progeny exhibits identical characteristics to the original plant. Asexual propagation by division and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWINGS

FIG. 1 shows a nine-month-old Heucherella ‘Solar Eclipse’, growing in a one gallon container in a cool greenhouse in May in Canby, Oreg.

FIG. 2 shows the foliage color of a one and a half year old Heucherella ‘Solar Eclipse’ grown in the shade in late summer in Canby, Oreg.

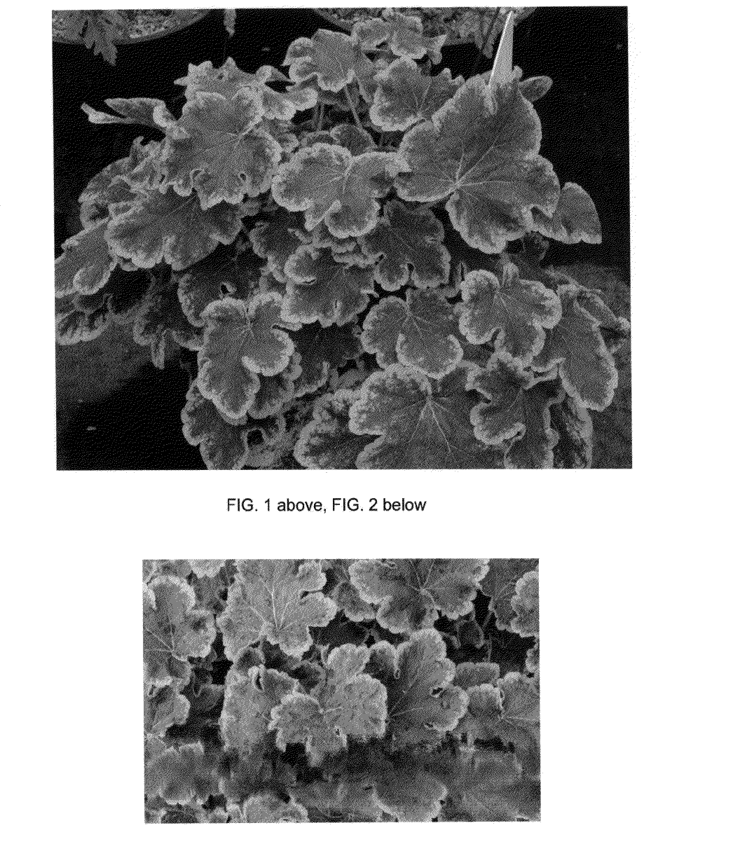

### DETAILED PLANT DESCRIPTION

The following is a detailed description of the new Heucherella cultivar based on observations of a one and a half-year-old specimen growing in a 2 gallon container in a cool greenhouse in Canby, Oreg. Canby is Zone 8 on the USDA Hardiness map. Temperatures range from a high of 95 degrees F. in August to an average of 32 degrees F. in January. Normal rainfall in Canby is 42.8 inches per year. The color descriptions are all based on The Royal Horticultural Society Colour Chart, 5^{th} edition.
Plant:
   Form.—basal rosette, herbaceous perennial.
   Hardiness.—USDA Zone 4 to 9.
   Size.—20 cm tall from top of the foliage to the ground and 50 cm wide.
   Habit.—mounding.
   Vigor.—excellent.
   Roots.—fibrous, freely branching, fine, and white in color.
Leaf:
   Type.—simple.
   Arrangement.—rosette.
   Shape.—ovate.
   Lobing/division.—7 palmate rounded lobes with the terminal the longest, the terminal lobe with shallow secondary lobes.
   Venation.—palmate.
   Margins.—crenate, slightly undulate.
   Apex.—mucronulate.
   Base.—cordate, overlapping at the base.
   Blade size.—grows to 10 cm long and 9 cm wide.
   Surface texture.—glandular both surfaces.
   Petiole description.—grows to 18 cm long and 3 mm wide, glandular hairs, color between Greyed Purple 183D and 186D on bottom half and lightening to between Yellow Green 148C and 152D.
   Leaf color.—new leaves in spring and fall topside Greyed Purple 187A with leaf margin Yellow Green N144C; summer, winter, and mature leaves topside Greyed Purple N187A with the margin Yellow Green 146B.
Inflorescence:
   Size.—5 cm wide and 12 cm long.
   Type.—raceme.
   Number of flowers per raceme.—about 70.
   Number of racemes.—about 35 in first spring flush.
   Peduncle.—grows to about 44 cm tall, 3 mm wide at base, Yellow Green 146C, glandular hairs, with 0-2 branches with cauline leaves at the base (cauline leaves petiolate, blades 4 cm long and 3.5 cm wide, ovate, sometimes palmately lobed, margins broadly crenate, Yellow Green N144C with leaf center irregularly splattered Greyed Purple 187A).
   Pedicel.—grows to 12 mm long, with glandular hairs, Greyed Red 182B.
   Bloom period.—April to July in Canby, Oreg.
Flower bud:
   Size.—1.5 mm wide and 3 mm long.
   Description.—glandular puberulent, ovoid.
   Color.—NN155A.
Flower:
   Type.—perfect.
   Shape.—campanulate.
   Size.—5 mm deep and 7 mm wide.
   Petal description.—5 in number, oblanceolate, 35 mm long and 1 mm wide, tip acuminate, base attenuate, margin entire, glabrous on both sides, White NN155D on both sides.
   Calyx description.—3 mm deep and 5 mm wide, with 5 lobes, divided 1/2 way to the base, campanulate, glandular hairs on outside, glabrous inside, tip obtuse, margin entire, White NN155C on both sides.
   Stamen description.—5 in numbers, filaments 3 mm long, White NN155D, anthers sterile, less than 1 mm, Greyed Orange 164A.
   Pistil description.—one central two beaked pistil, 4 mm long, White 155B.
   Fragrance.—none.
   Lastingness.—each raceme blooms for about 2 weeks on the plant.
Fruit and seed: none
Disease and pest tolerance: No known resistances. Excellent disease tolerance to powdery mildew. All Heucherella are susceptible to root weevils.

We claim:

1. A new and distinct Heucherella plant as herein illustrated and described.
